(19) United States
(12) Patent Application Publication
(10) Pub. No.: US 2023/0272321 A1
(43) Pub. Date: Aug. 31, 2023

(54) INSERT CHIP AND A SYSTEM COMPRISING SAME FOR CELL CULTURE

(71) Applicant: Ramot at Tel-Aviv University Ltd., Tel-Aviv (IL)

(72) Inventors: Ben Meir MAOZ, Tel-Aviv (IL); Rossana RAUTI, Tel-Aviv (IL); Adi ESS, Tel-Aviv (IL); Mark Dan KIRI, Tel-Aviv (IL)

(73) Assignee: Ramot at Tel-Aviv University Ltd., Tel-Aviv (IL)

(21) Appl. No.: 18/144,937

(22) Filed: May 9, 2023

Related U.S. Application Data
(63) Continuation of application No. PCT/IL2021/051317, filed on Nov. 8, 2021.

(30) Foreign Application Priority Data
Nov. 9, 2020 (IL) 278594

(51) Int. Cl. C12M 3/06; C12M 1/12; C12M 1/00; C12M 1/42

(52) U.S. Cl. CPC C12M 23/16; C12M 25/04; C12M 25/14; C12M 29/04; C12M 35/02

(57) ABSTRACT

Provided herein is an insert chip and a cell culture system including the same, adapted for culturing a plurality of cell populations under various flow patterns.

### RELATED APPLICATIONS

This application is a Continuation of PCT Patent Application No. PCT/IL2021/051317 having International filing date of Nov. 8, 2021, which claims the benefit of priority of Israeli Patent Application No. 278594 filed on Nov. 9, 2020. The contents of the above applications are all incorporated by reference as if fully set forth herein in their entirety.

### FIELD OF THE INVENTION

Provided herein is an insert chip and a cell culture system including the same, adapted for culturing a plurality of cell types under various conditions.

### BACKGROUND

The development of in vitro models that recapitulate in-vivo features is essential for elucidating human physiology and disease mechanisms, as well as for drug discovery. As human physiology is highly complex, such in vitro models should ideally take many parameters into account, including the following: cellular microenvironment, cell-cell communication, organ-organ interactions, and mechanical aspects such as hydrodynamic and shear stress, which are critical for the development of cellular functionality. In recent years, several in vitro modeling platforms have been developed with the capacity to capture many of these features. These platforms include Transwell (TW) cell culture inserts, which enable cells to be co-cultured over a membrane microfluidic device (Organs-on-a-Chip), which allow for both co-culturing and the application of flow and other mechanical forces organoids, which mimic 3D tissue structure; and other 3D-systems that recreate a 3D microenvironment.

Several studies have tried to combine different in vitro modeling approaches to overcome the challenges outlined above. Sip et al. (Lab Chip, 2014, 14: 302-314), for example, developed a TW with flow, which uses soft-lithography to produce PDMS microchannels which are attached to 6-well TW holders. International Patent Application Publication No. WO 2018/020274 is directed to blood brain barrier model. International Patent Application Publication No. WO 2019/219605 is directed to method for the preparation of a cell culture insert with at least one membrane.

Though these platforms somewhat constitute recapitulating in vivo environments, each has shortcomings that hinder its universal application.

There is thus a need in the art for improved insert chips that are cost effective, versatile, reusable and reliable, that can accommodate various cell populations and simulate different growth conditions.

### SUMMARY OF THE INVENTION

According to some embodiments, there is provided herein an advantageous insert chip (also termed herein “Insert-Chip”) and a system including same, for culturing a plurality of different cell types.

Advantageously, the insert chip and system disclosed herein provide a modular platform which is cost efficient, easy to use, applicable to various high-throughput experiments, capable of capturing cell-cell interactions, capable of inducing flow, and compatible with high-magnification imaging procedures.

According to some embodiments, the disclosed insert-chip is a modular, inexpensive, and user-friendly chip that exposes cultured cells to a controllable flow, and that can support cell-cell interactions and co-cultures. Advantageously, the Insert-Chip can be integrated into a variety of standard well plate cell culture platforms (and/or MEA platforms.

According to some embodiments the insert chip disclosed herein provides an innovative Organ-on-a-Chip platform that can be easily fabricated (for example, with 3D printing, as detailed herein) and be integrated or used with standard cell culture systems.

According to some embodiments, as exemplified herein, the Insert-Chip has the capacity to allow the growth of different types of cells (for example, endothelial cells, epithelial cells, neuronal cells, cancer cells, and the like) under different flow patterns and/or other forces (such as, shear force), and to provide straightforward access to various types of measurements that are of importance in physiological and drug development studies, including, for example, barrier permeability.

According to some embodiments, as exemplified herein, the cell culture system disclosed herein can allow simultaneous culturing of at least two, at least three spatially distinct cell populations (i.e., the cells are physically separated).

According to some embodiments, the advantageous modularity of the Insert-Chip, coupled with its capacity to enable multiple cell-types to be co-cultured and observed under various conditions (such as, flow and shear), can simplify experimental procedures that are currently highly complex in in-vitro studies in academic and industry settings. In particular, the Insert-Chip device can facilitate the study of cell-cell interactions, such as neurovascular coupling, essential to understanding the pathogenesis of multiple diseases.

There is provided, according to some embodiments, an insert chip for cell culture, the insert chip includes:
a. a hollow scaffold adapted to enclose therewithin a porous membrane, wherein the hollow scaffold comprises at least one inlet configured to deliver a first fluid thereinto and at least one outlet configured to withdraw/remove fluids therefrom and wherein each of said at least one inlet and at least one outlet is configured to fluidly associate with a corresponding first fluid receptacle and withdrawn fluid receptacle, respectively, through corresponding conduits; and
b. a porous membrane having an upper side and a lower side, the porous membrane is configured to accommodate cell culture on each side, wherein the porous membrane is configured to be positioned/held between inner walls of the hollow scaffold. In some embodiments, the porous membrane is configured to be positioned at the bottom portion of the hollow scaffold and may be attached to an inner surface thereof.

According to some embodiments, the insert chip may further include a support structure, such as, in the form of a support ring, configured to support the porous membrane within the hollow scaffold (i.e., between inner walls thereof).

According to some embodiments, the support ring may be made of silicone, such as, for example, polydimethylsiloxane (PDMS). In some embodiments, the support ring may be associated with the membrane prior to the membrane being placed/located in the hollow scaffold. In some embodiments, the support structure maybe placed on the membrane after the membrane has been placed/positioned/located in the hollow structure.

In some embodiments, in addition to or instead of the support structure, a reducer element (insert-reducer) may be placed on the membrane, to reduce the surface area thereof, as further detailed below herein. In some embodiments, the reduced element may be made of silicone, such as, PDMS. In some embodiments, the reducer element may have a linear shape, curved shape, or the like.

According to some embodiments, the insert chip may further include a plurality of legs (pillars) extending from the bottom part/portion of the hollow scaffold and configured to position the hollow scaffold on a flat surface such that the distance between the porous membrane and the flat surface is determined by the length of the plurality of pillars. In some embodiments, the length/height of the pillars is adjustable.

According to some embodiments, the hollow scaffold may be made of a transparent material, such as, for example, a transparent polymer.

According to some embodiments, the hollow scaffold may be made of a dental resin.

There is provided, according to some embodiments, a cell culture system which includes:
i. at least one cell culture container comprising the insert chip disclosed herein, wherein the porous membrane is positioned within the insert chip such that the lower side thereof is facing the bottom surface of the at least one cell culture container, and is detachable therefrom;
ii. at least one first fluid receptacle fluidly associated with the at least one inlet; and
iii. at least one withdrawn fluid receptacle fluidly associated with the at least one outlet.

According to some embodiments, the cell culture system may further include at least one inlet conduit fluidly connecting the at least one inlet to the at least one first fluid receptacle; and at least one outlet conduit fluidly connecting the at least one outlet to the at least one withdrawn fluid receptacle.

According to some embodiments, the at least one cell culture container may be configured to contain/hold fluids at a fluid level such that the fluids contact the porous membrane, wherein the fluids include a fresh first fluid flowing therein through the at least one inlet and withdrawn therefrom through the at least one outlet at a flow rate adjusted to maintain the fluids level within the cell culture container.

According to some embodiments, the cell culture system may include a plurality of cell culture containers and a plurality of insert chips as disclosed herein, each insert chip is contained/placed/held within a cell culture container.

According to some embodiments, the plurality of cell culture containers may be selected from a multi-well plate and multi-electrode array (MEA) environment.

According to some embodiments, the first fluid is a tissue culture medium.

According to some embodiments, there is provided ab insert chip for cell culture, the insert chip includes:
a. a hollow scaffold adapted to enclose therewithin a porous membrane, wherein the hollow scaffold includes at least one inlet configured to deliver a fluid thereinto and at least one outlet configured to withdraw fluids therefrom and wherein each of said at least one inlet and at least one outlet is configured to fluidly associate with a corresponding fluid receptacle, through corresponding conduits;
b. the porous membrane having an upper side and a lower side, and is configured to accommodate cell culture on each side thereof, wherein the porous membrane is configured to be positioned at a lower part/portion of the hollow scaffold; and
c. one or more legs extending from a bottom part/portion of the hollow scaffold and configured to position the hollow scaffold on a surface such that the distance between the lower side of the porous membrane and the flat surface is determined by the length of the plurality of legs.

According to some embodiments, the insert chip may further include a support ring, configured to support the porous membrane within the hollow scaffold.

According to some embodiments, the length of the one or more legs may be adjustable.

According to some embodiments, the insert may include a plurality of legs, wherein the length of the legs may essentially similar.

According to some embodiments, the membrane may be associated with an inner surface of the hollow scaffold.

According to some embodiments, the membrane is at least partially held between inner walls of the hollow scaffold.

According to some embodiments, the membrane may be removable.

According to some embodiments, the insert chip may further include an insert-reducer configured to reduce a surface area of the membrane on which the insert-reducer is positioned/placed on.

According to some embodiments, the insert chip may include two inlets and two outlets. According to some embodiments, each set of inlet and outlet is configured to allow passage of a separate fluid. According to some embodiments each set of inlet and outlet is configured to provide fluid flow to/over a distinct compartment of the insert chip.

According to some embodiments, the insert chip is configured to allow controlling or determining flow pattern, flow strength and/or shear forces applied on cells associated therewith.

According to some embodiments, least the hollow scaffold of the insert chip is essentially transparent.

According to some embodiments, at least the hollow scaffold is reusable.

According to some embodiments, the insert is configured to be placed or located in a culture well plate and/or multi-electrode array (MEA).

According to some embodiments, the cells may be selected from primary cells, culture cells, endothelial cells, epithelial cells, neuronal cells, cancer cells, or any combination thereof.

According to some embodiments, there is provided a cell culture system which includes:
i. at least one cell culture container comprising an insert chip as disclosed herein, wherein the porous membrane is positioned within the insert chip such that the lower side thereof is facing the bottom surface of the at least one cell culture container, and is detachable therefrom;
ii. at least one first fluid receptacle fluidly associated with the at least one inlet; and
iii. at least one withdrawn fluid receptacle fluidly attached to the at least one outlet.

According to some embodiments, the cell culture system may further include at least one inlet conduit fluidly connecting the at least one inlet to the at least one fluid receptacle; and at least one outlet conduit fluidly connecting the at least one outlet to the at least one withdrawn fluid receptacle.

According to some embodiments, the at least one cell culture container is configured to contain fluids at a fluid level such that the fluids contact the porous membrane, wherein the fluids include a fresh first fluid flowing therein through the at least one inlet and withdrawn therefrom through the at least one outlet at a flow rate adjusted to maintain the fluids level within the cell culture container.

According to some embodiments, the cell culture system may further include one or more pumps configured to allow fluid passage between the conduits.

According to some embodiments, the cell culture system may include a plurality of cell culture containers.

According to some embodiments, at least a portion of the plurality of cell culture containers hold/harbor an insert chip.

According to some embodiments, each of the plurality of cell culture containers comprises an insert chip.

According to some embodiments, wherein at least a portion of the insert chips in the plurality of cell culture containers are fluidly connected.

According to some embodiments, the cell culturing system is configured to allow essentially simultaneous culturing of at least two distinct cell populations.

According to some embodiments, the cell culturing system is configured to allow essentially simultaneous culturing of at least three spatially distinct cell populations.

According to some embodiments, the distinct cell populations are physically and/or spatially separated.

According to some embodiments, the cell populations may be selected from: primary cells, culture cells, endothelial cells, epithelial cells, neuronal cells, cancer cells, or any combination thereof

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIGS. 1A-D show illustrations of insert-chip, according to some embodiments. FIG. 1A shows a schematic illustration of a close up view of components of an exemplary insert having one inlet and one outlet; FIG. 1B shows a schematic illustration of a close up view of components of an exemplary insert having two distinct sets of inlets and outlets, each configured to allow fluid passage in/over a different compartment of the insert chip. Left hand panel shows a partial cross section of the insert chip, illustrating fluid flow in the first set of inlet-outlet and the second set of inlet-outlet; FIG. 1C shows a schematic illustration of an experimental design, whereby the Insert-Chip can be placed in a cell culture platform (for example, culture well plate). In the schematic, one type of cells are grown on the top of the porous membrane inside the Insert-Chip, while other types of cells are grown on the bottom of a well-plate; FIG. 1D shows a pictogram of an assembled Insert-Chip integrated in a petri dish (culture well), with two different colored solutions: one (red) inside the chip and the other (blue) on the bottom of the culture plate;

FIGS. 2A-B represents modularity and versatility of the Insert-Chip, according to some embodiments. FIG. 2A shows images of Insert-Chip with different leg (pillars) heights (LH; 1 mm, 2 mm, and 4 mm, respectively); FIG. 2B shows time series of the diffusion simulations results: Cross section view of the reduced chip with 4 mm (a) and 1 mm (b) LH showing CO₂ accumulation at the bottom of the container.

FIGS. 3A-C demonstrate the versatility of the Insert-Chip, according to some embodiments. FIG. 3A shows illustration of respective pictogram of Insert-Chips fabricated in different sizes in order to be associated with commercially available 6, 12, and 24 well-plates (from left to right); FIG. 3B shows a pictogram of 6 Insert-Chips, each placed in a separate well placed in a 12-well plate and linearly connected to each other, in order to simulate multi-organ-chip platforms; FIG. 3C shows a pictogram of magnification of two Insert-Chips placed in distinct wells and connected under flow;

FIG. 4 shows an exemplary Insert-Chip fabrication process, according to some embodiments. Shown in FIG. 4 is a schematic time-line representation of the Insert-chip and the ring (support structure) fabrication followed by the chip assembly.

FIGS. 5A-F show an exemplary epithelial and endothelial barrier, grown on the Insert-Chip. FIG. 5A shows Confocal reconstructions pictograms of epithelial (Caco-2) cells immunostained for ZO-1 (green) and nuclei (DAPI); FIG. 5B shows Confocal reconstructions of endothelial (HUVEC) cells immunostained for CD31 (green) and nuclei (DAPI); FIG. 5C shows graphs Plot showing pooled TEER (Trans-epithelial endothelial electrical resistance) values of Caco-2 cells; FIG. 5D shows HUVEC cells cultured on the Insert-Chip with and without flow and on Transwells (TW); FIG. 5E shows Relative Permeability Values of Caco-2; FIG. 5F shows HUVEC cells measured as leakage of FITC-dextran from the upper to the bottom compartment of the Insert-Chip;

FIGS. 6A-B show high-resolution imaging of cells cultured in an Insert-Chip, according to some embodiments. FIG. 6A shows a schematic design (from left to right) of the removal of the porous membrane containing/harboring cultured cells from the insert-chip, to be placed on a microscopic cover slip, in order to perform high-resolution confocal imaging. FIG. 6B shows pictograms of Confocal reconstructions at 60× magnification of HUVEC cells cultured on the porous membrane and stained for CD-31 (green) and DAPI (blue);

FIGS. 7A-E show various types of insert-reducers (linear and “S”-shaped), configured to control flow, according to some embodiments. FIG. 7A shows a schematic experimental design of a linear insert-reducer (“reducer”) that enables flow and shear stress to be controlled. FIG. 7B show graph plot showing shear stress values at different flow rate, changing the width of the insert-reducer. FIG. 7C shows CFD calculated flow streamlines at a constant flow rate of 5 μL/min through the insert chip with the reducer (a.1) and without (a.2) a reducer; CFD calculated WSS map at a constant flow rate of 5 μL/min through the chip (b.1) with the reducer surface walls and membrane showing less than 0.025 dyne/cm² on the membrane (max=0.021 dyne/cm²) (b.2). WSS on the chip without reducer, showing less than 0.025 dyne/cm² on the membrane (max=0.0023 dyne/cm²); CFD calculated WSS map at a constant flow rate of 50 μL/min through the chip (c.1); WSS on the chip with the reducer, showing a significantly higher shear with 0.22 dyne/cm² maximum shear. (c.2) WSS on the chip without the reducer, showing more than 0.025 dyne/cm² on the walls (max=0.027 dyne/cm²), which is higher than the WSS for the case with reducer and a flow rate of 51.μL/min; FIG. 7D shows a pictogram of the reducer associated with the Insert-Chip and connected to an external pump with red color flushed inside (left hand panel). In the right panel, confocal tile scan reconstructions of Caco-2 cells grown under flow and able to form a confluent monolayer in the channel, immunostained for DAPI (blue) and ZO-1 (green); FIG. 7E shows pictogram of an “S”-shaped reducer (left hand panel). Right hand panel shows HUVEC cells grown inside the channel and immunostained for CD-31 (green) and DAPI (blue);

FIGS. 8A-D Utilizing the Insert-Chip with MEA (multi-electrode array) system, according to some embodiments. FIG. 8A shows a pictogram showing the Insert-Chip associated with the MEA platform, allowing for simultaneous TEER and electrophysiological measurements. FIG. 8B shows pictograms of Rat hippocampal neurons cultured on the MEA device for 10-12 days in vitro. FIG. 8C shows TEER graph plot of HUVEC cells cultured on the Insert-Chip and integrated/associated with the MEA. FIG. 8D shows extracellular electrophysiological recordings of neuronal spontaneous activity recorded from 14 different electrodes after 10 days in vitro, simultaneously integrated with HUVEC grown on the Insert-Chip. Each color represents a different electrode;

FIG. 9 Culturing a plurality of cell populations utilizing an insert-chip culturing system, according to some embodiments. Left hand panel shows a schematic illustration of the insert chip in a culture well, showing three different cell types/populations can be cultured utilizing the Insert-Chip (on top of the membrane, on the bottom of the membrane, and on the bottom of the well). Right hand panel shows pictograms of confocal reconstruction showing a 3D reconstruction of the culturing system, with enlarged images of SY-SH5Y (stained for actin in green and DAPI in blue), grown on the well-plate; U87 (stained for GFAP in red and DAPI in blue) on the top of the membrane; and HUVEC (stained for CD31 in green and DAPI in blue) on the bottom of the membrane; and

FIGS. 10A-B Schematic of exemplary flow patterns/circulations utilizing an Insert-Chip, according to some embodiments. FIG. 10A show schematic illustrations of various exemplary flow circulations that can be achieved using the Insert-Chip, depending on the linkage setting of a pump; FIG. 10B shows schematic illustrations of various exemplary flow circulations, using a “reduced” Insert-Chip (i.e., utilizing an insert-reducer). Different flow circulations can be achieved using the Insert-Chip with a reducer, depending on the linkage setting of the pump.

### DESCRIPTION OS SPECIFIC EMBODIMENTS OF THE INVENTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

In the description and claims of the application, the words “include” and “have”, and forms thereof, are not limited to members in a list with which the words may be associated.

According to some embodiments, organ-on-a-Chip platforms provide rich opportunities to observe interactions between different cell types under in vivo-like conditions, i.e., in the presence of flow. Yet, the costs and knowhow required for the fabrication and implementation of these platforms restrict their accessibility. The advantageous Insert-Chip disclosed herein is a microfluidic device that provides the functionality of an Organ-on-a-Chip platform, namely, the capacity to co-culture cells, expose the cells to flow (e.g. of culture media), and enables to observe cell-cell interactions, yet it can be easily integrated/be used with standard culture systems (e.g., well plates or multi-electrode arrays (MEA)). According to some embodiments, as detailed herein, the insert chip may be produced using stereolithograpy 3D printing and is user-friendly and reusable. Moreover, its design features overcome some of the measurement and imaging challenges characterizing standard Organ-on-a-Chip platforms. The advantageous Insert-Chip device and its capabilities are exemplified with exemplary cultures of endothelial and epithelial cells, which are co-cultured with neuronal cells, subjected to flow, and analyzed with various assays. Overall, the microfluidic device disclosed herein is a valuable platform for the investigation of biological functions, cell-cell interactions, response to therapeutics, and the like.

According to some embodiments, as further exemplified herein below, the advantageous insert chip can be used to perform experiments with various types of cells at various maturation stages, while allowing to simultaneously monitor various cellular functionalities even if characterized by different maturation times.

According to some embodiments, as further exemplified herein below, the advantageous insert chip can be used to form and/or study various cellular barriers. In some embodiments, utilizing the insert chip system, cells can create a barrier layer, and flow induction can enhance the barrier properties.

According to some embodiments, the insert-chip platform disclosed herein is close to “ideal” as it combines the strengths of two popular platforms, namely, Transwells (TWs) and the Organ-on-a-Chip, while overcoming some of their limitations.

TW inserts are commercially available in a range of size, easy to use, and can be used as a high-throughput tool. Yet, TWs are considered to be “static” models, as they do not have the capacity to induce flow, a crucial feature for models of vasculature and epithelial tissues. The Organ-on-a-Chip, in turn, enables flow to be induced, and can provide insight regarding organ-organ interactions; however, Organ-on-a-Chip systems are not modular, and their fabrication and implementation typically require a great deal of time and knowhow. Moreover, most chips are made of polydimethylsiloxane (PDMS), which adsorbs hydrophobic compounds, limiting the platform's applicability to drug testing. An additional shortcoming, shared by both TW and Organ-on-a-Chip systems, is the substantial difficulty in using high-resolution microscopy to investigate cell dynamics, owing to the large working distance needed for visualizing the cells.

According to some embodiments, to capture the benefits of TW inserts and Organs-on-a-Chip, while overcoming their individual and shared limitations, the advantageous insert-chip platform disclosed here utilizes new fabrication tools (3D printing) to develop an easy-to-use, customizable, microfluidic chip that, can be inserted into any standard culture platform, thereby transforming it into an advanced in vitro model platform.

Thus, according to some embodiments, there is provided an insert chip for cell culture, the insert chip includes: a hollow scaffold adapted to enclose therewithin a porous membrane, wherein the hollow scaffold comprises one or more inlets configured to deliver a fluid thereinto (or to portions/compartments thereof) and one or more outlets configured to withdraw fluids therefrom, and wherein each of said one or more inlets and one or more outlets is configured to be fluidly associated with corresponding fluid receptacle(s) and withdrawn fluid receptacle(s), respectively, through corresponding conduits; and a porous membrane having two sides, an upper side and a lower side, the porous membrane is configured to/capable of accommodating cell culture population on each side, wherein the porous membrane is positioned at a lower portion/part/region (i.e., closer to the bottom part/portion) of the hollow scaffold and is attached to an inner surface thereof. In some embodiments, the membrane is associated with a support structure (such as, a ring) that may be placed on at least a portion of the top surface of the membrane.

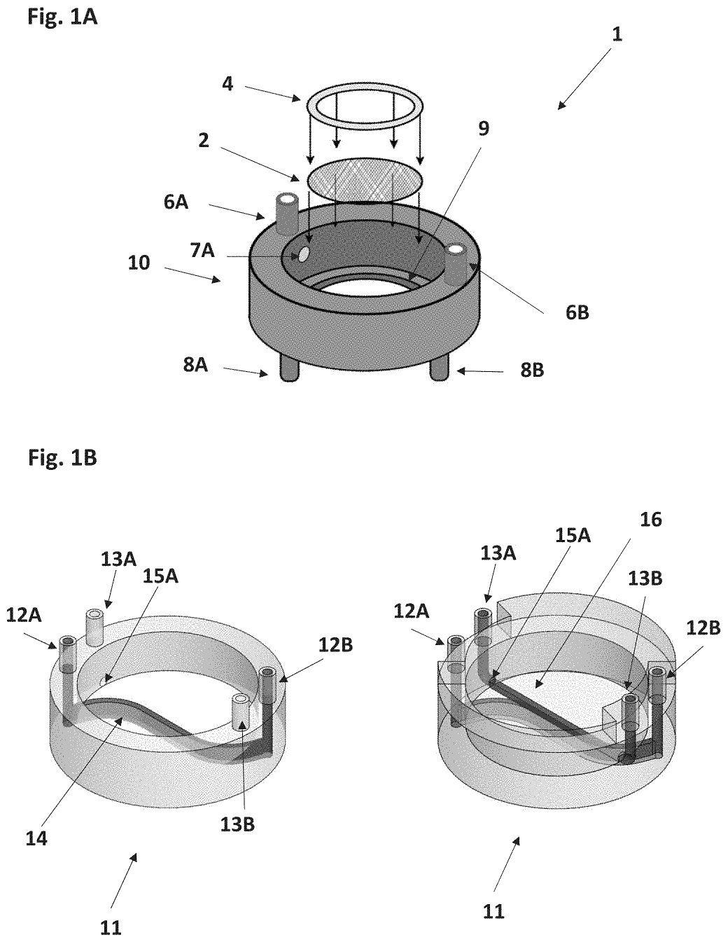

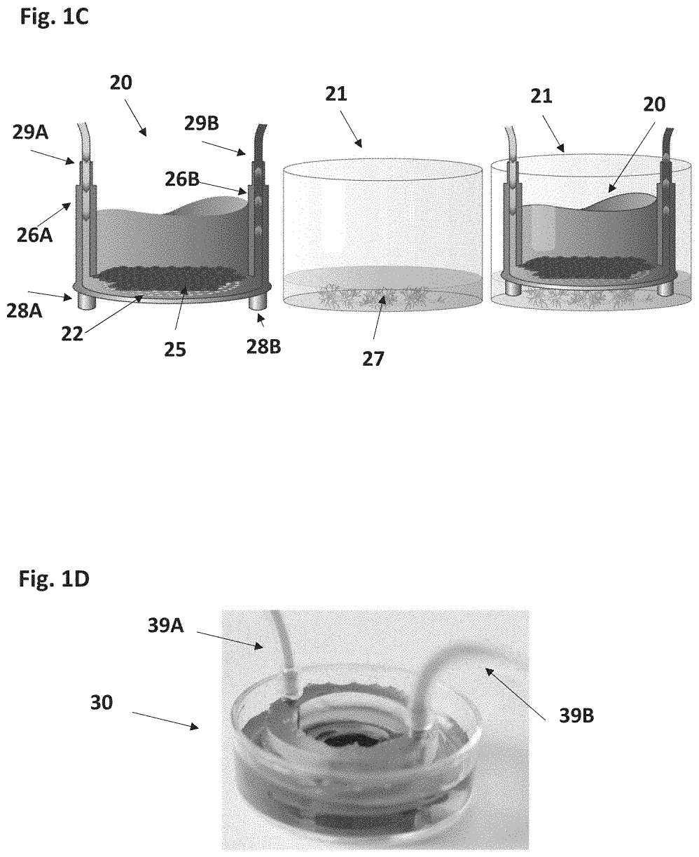

Reference is now made to FIG. 1A, which shows a schematic illustration of a close up view of components of an exemplary insert having one inlet and one outlet. As shown in FIG. 1A, insert chip 1 includes a hollow scaffold (body) 10, having a top portion and a lower portion (configured to face a culture well bottom surface). The hollow scaffold body may be fabricated in any desired shape and size, such as, for example, a cylinder having dimensions (for example, external and internal diameter) to fit in a corresponding culture well. As shown in FIG. 1A, hollow scaffold 10 may include an optional internal rim 9, configured to hold/position membrane 2, when the membrane is placed therein. Further shown is support element 4, shown, for example, in the form of a ring. Support element 4 is configured to be placed on a top surface of membrane 2 and may aid is stabilizing its structure, aligning the membranes into its location/position in the hollow scaffold and/or further provide sealing. Hollow scaffold 10 further includes an inlet 6A and a corresponding outlet 6B, each having an opening external to the hollow scaffold and an opening/aperture in the wall of the hollow scaffold (wall opening 7A of inlet 6A is shown in FIG. 1A). Further shown are pillars/legs/extensions 8A-8B, of the hollow scaffold, that can allow adjusting the height/distance of the insert chip from the bottom surface of the well plate. In some embodiments, the legs height/length is adjustable.

Reference is now made to FIG. 1B, which shows a schematic illustration of a close-up view of components of an exemplary insert having two distinct sets of inlets and outlets, each configured to allow fluid passage in/over a different compartment of the insert chip. As shown in the Left hand panel of FIG. 1B, hollow scaffold 11 includes two sets of inlets and outlets: inlet 12A is associated with outlet 12B and is fluidly connected via a suitable conduit 14. A second set includes inlet 13A and outlet 13B. Further shown is opening/aperture 15A of inlet 13A, in the wall of the hollow scaffold. Right hand panel of FIG. 1B shows a partial cross section of the insert chip hollow scaffold 11 and further showing conduit 16 allowing fluid connection between inlet 13A and outlet 13B. As illustrated in FIG. 1B, two distinct fluid flows can thus be formed, in separate compartments of the insert chip, namely, a fluid flow in the first set of inlet-outlet (for example, 12A-12B via conduit 14) in an upper compartment, and the second set of inlet-outlet (for example, 13A-13B, via conduit 16), in a lower compartment.

According to some embodiments, such an advantageous setting as illustrated in FIG. 1B, the Insert-Chip includes two different/distinct channels with the option to differentially control the flow in each channel/environment. Tin some embodiments, such insert chip (including a membrane and supporting ring) may further be pressed/tightened to the bottom of well plate with a special/dedicated lid that can prevent leakage from the channels. By such arrangement of dual channel chip, the following advantages may be obtained: 1. Two dynamic environments can be created/used. The inclusion of another set of inlet/outlet to the bottom part, forms a dual-channel chip. In some embodiments, such a setting enables mimicking and experimenting in anaerobic conditions, as the thick layer of silicone (such as, PDMS) can hold a low-oxygen environment in one of the channels. Such experiments can be used to mimic tissues that requires anaerobic/hypoxic condition, such as the gut tract or various stroke model.

Reference is now made to FIG. 1C, which shows a schematic illustration of an experimental design, whereby an Insert-Chip can be placed in a cell culture platform (for example, culture well plate), according to some embodiments. As shown in FIG. 1C, insert chip has legs 28A-B, for adjusting its height over the bottom surface of culture well 21. Membrane 22 of insert chip 20 has cells 25 grown over the top surface thereof. Fluid is capable of being passed/flow between inlet 26A and outlet 26B, via corresponding conduits 29A-B. The bottom surface of culture well 21 has cells 27 grown thereon. As shown in the right hand panel of FIG. 1C, the insert chip, including cells 25 on membrane 22 is accommodate/placed/fitted within culture well 21, facilitating potential association or interaction between the two cell populations (i.e., cells 25 and cells 27).

Reference is now made to FIG. 1D which shows a photograph of an assembled Insert-Chip 30, placed in a petri dish, with two different colored solutions/fluids, one (red) inside the chip and the other (blue) on the bottom of the culture plate, wherein the first (red) solution/fluid is provided via conduits 39A-B.

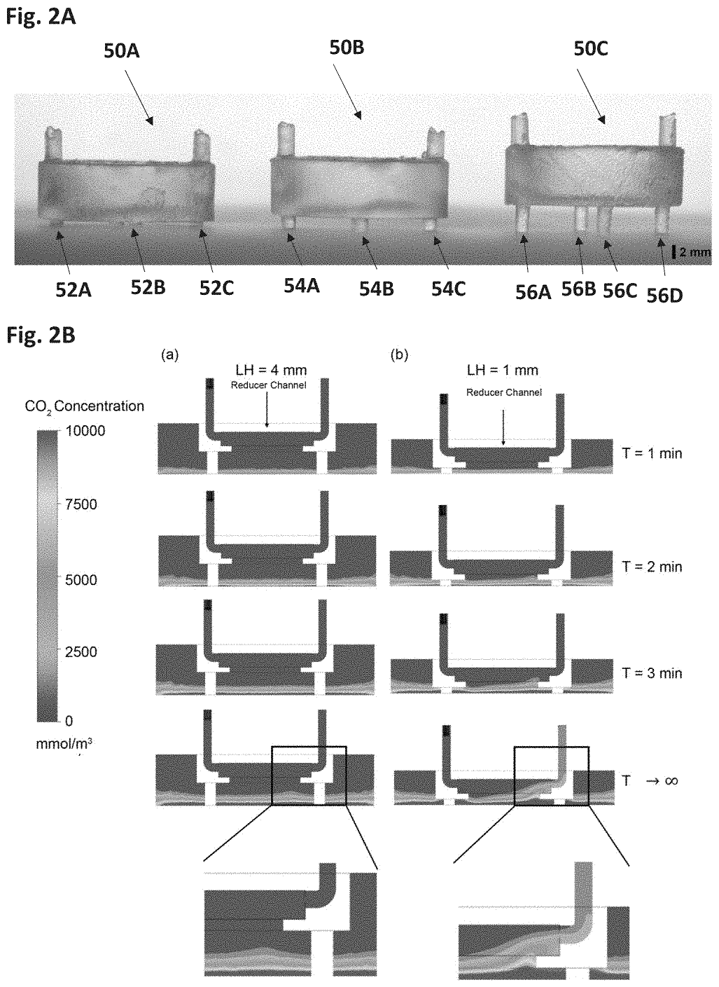

Reference is now made to FIG. 2A, which shows images of Insert-Chip with different leg (pillars) heights (“LH”; 1 mm, 2 mm, and 4 mm, respectively). As shown in FIG. 2A, insert chip 50A includes legs 52A-C, having a relatively short length (in this example, 1 mm). Insert chip 50B includes legs 54A-C, having a longer length (in this example, 2 mm). insert chip 50A includes legs 56A-C, having a relatively longer length (in this example, 4 mm). Adjusting or determining the height/length of the legs, allows changing the distance (height) between the membrane and the bottom plate. This versatility is especially important for controlling the diffusion, material gradient, and/or shear forces between the upper and lower compartments. FIG. 2B shows simulation of the diffusion of CO₂ in the Insert-Chip with 1 mm and 4 mm legs-height (LH), as further detailed herein below. According to some embodiments, the insert-chip comprises a plurality of pillars/legs/short legs. According to some embodiments, the length of each pillar in the plurality of pillars is within the range of about 0.5 to 10 mm. According to some embodiments, each pillar is about 1-2 mm long. According to some embodiments, the plurality of pillars are of the same length. According to some embodiments, the insert chip is configured to stand on the plurality of legs, and thus can stand alone in either a well plate or multi-electrode array (MEA) environment, above a cell culture surface, thereby enabling the cells in that environment to interact with the cells in the chip. According to some embodiments, the height of the legs is adjustable.

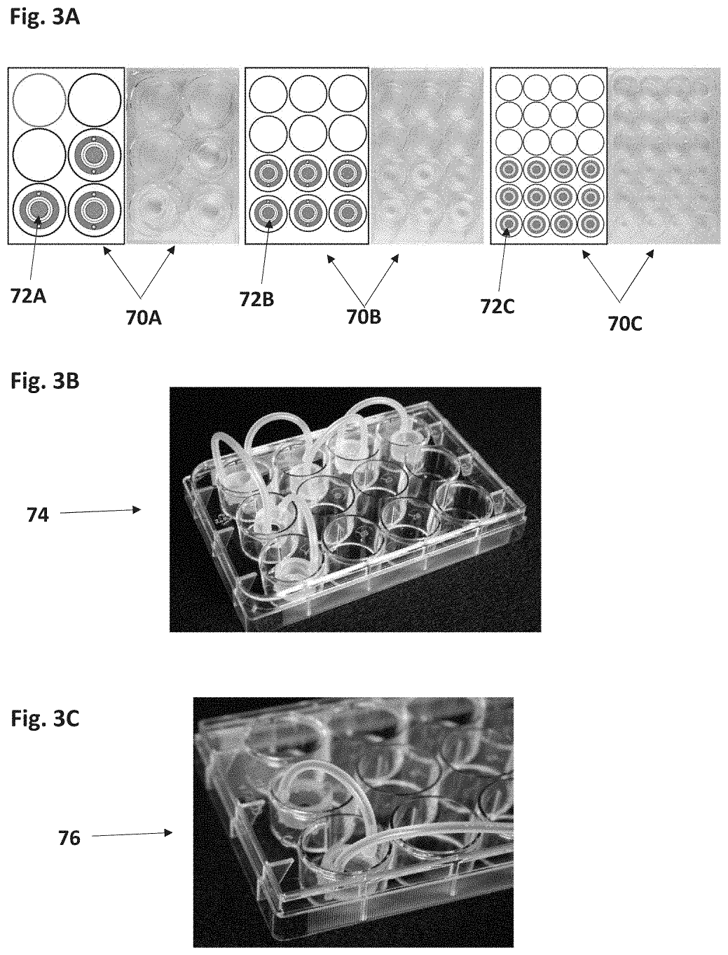

Reference is now made to FIG. 3A, which shows illustration of respective pictogram of Insert-Chips fabricated in different sizes in order to be associated with commercially well-plates. As shown in FIG. 3A, 6-well plate 70A includes 6 distinct culture wells. In at least half (three) of the wells, insert chips (such as exemplary insert chip 72A) are placed. 12-well plate 70B includes 12 distinct culture wells. In at least half (six) of the wells, insert chips (such as exemplary insert chip 72B) are placed. 24-well plate 70C includes 24 distinct culture wells. In at least half (12) of the wells, insert chips (such as exemplary insert chip 72B) are placed. The image shown in FIG. 3B is of an exemplary 12-wall plate 74, including 6 Insert-Chips, each placed in a separate well of plate 74, and are linearly connected to each other via corresponding conduits/pipes/tubes, in order to simulate cells-cross talk interactions (i.e. multi-organ-chip platforms). The image shown in FIG. 3C is a magnification of two Insert-Chips placed in distinct wells of 12-well plate 76, which are fluidly connected.

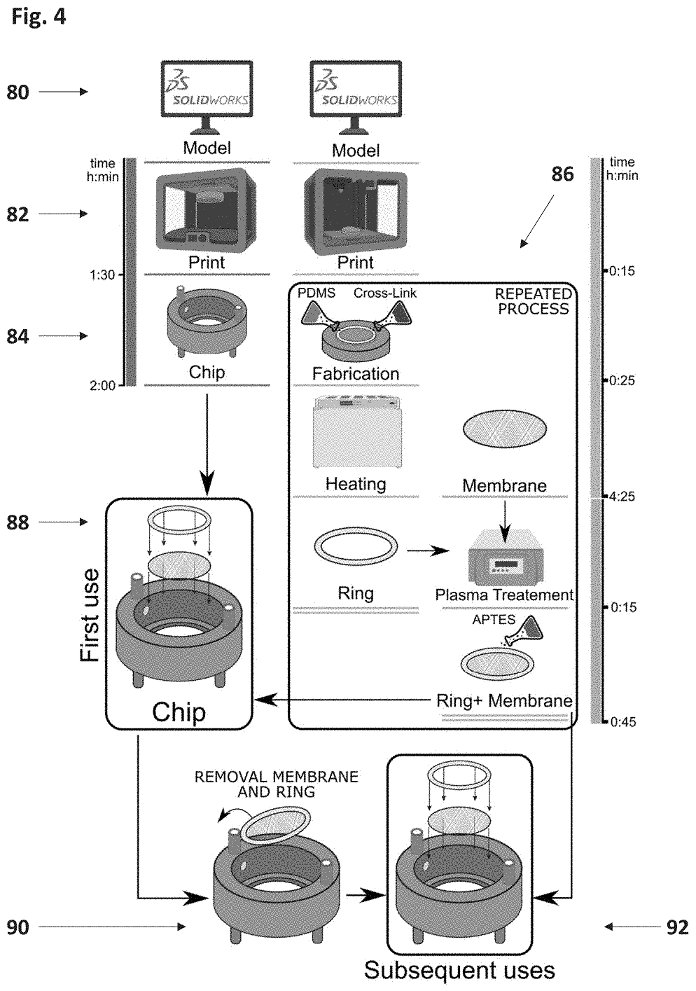

Reference is now made to FIG. 4 which shows an exemplary Insert-Chip fabrication process, according to some embodiments. Shown in FIG. 4 is a schematic time-line representation of the process of making an Insert-chip and a corresponding support structure. As shown in FIG. 4, at step 80, the properties of the insert chip are determined based on the intended application (affected, for example, based on the type of cells, type of cellular interactions tested, type and size of culture well, and the like). Such properties, include, for example, compositions, shape and/or size (such as external diameter, internal diameter, length, height, number of outlets/inlets, and the like). In step 82, the insert chip hollow scaffold is fabricated, for example, by printing. The chips may be printed, for example in a stereolithography Form2 3D printer, using suitable polymer, such as, for example, a dental long-term (LT) clear resin. After printing, at step 84, the formed hollow scaffold may be washed (for example, in isopropyl alcohol, in an ultrasound tank), to remove unreacted polymer/resin, and may then be cured and dried, for example, using a UV curing system. Sequentially, or in parallel, at step 86, one or more additional components of the insert chip may be made, including, for example, the membrane, the supporting structure/element (such as in the form of a ring), optional insert reducers, and the like. In some embodiments, a master-molds for fabrication of the additional components, such as, the supporting element (for example, in the form of PDMS support ring), and various types of “reducer” components aimed at reducing the active surface area in the chip and controlling the flow ((“insert-reducer”), may be prepared. The molds may be printed with a suitable filament, such as, for example, polylactic acid filament using a 3D printer (for example, Raise 3D Pro2 Dual Extruder 3D Printer). The prepared molds may then be filled with suitable material (such as, silicone, PDMS). The resulting PDMS supporting elements (rings) and reducers may then be cleaned dried at room temperature, and optionally activated in oxygen plasma. In addition, suitable membrane may be prepared. The suitable membrane may be fabricated or obtained in accordance with the experimental requirements (for example, type of cells, size of insert, size of culture well, and the like). In some embodiments, suitable Polycarbonate (PC) membranes (for example, having a pore size of between 0.01-1 μm (such as, 0.4 μm) pore and a thickness of, for example, 1-250 μm (such as, 25 μm)), may be adjusted to size of the insert chip hollow scaffold. The PC membranes may optionally be rinsed, dried activated and immersed in a suitable solution in order, for example, to coat the membrane and/or to introduce amino groups at the surface of the PC membrane. Thereafter, the produced supporting structure (such as, PDMS ring, or PDMS-reducers) and the corresponding membranes may be aligned and contacted, for example, by applying pressure, to ensure conformational contact. The association between the membrane and supporting structure/reducer may then be strengthened, for example, by baking, or otherwise increasing contact strength between the elements. Next, at step 88, the assembled parts (membrane+supporting structures (ring and/or reducer)) may be placed/positioned in the 3D-printed microfluidic Insert-Chip hollow body. The so-formed ready-to-use assembled insert chip may be further sterilized (for example, by ethanol wash and/or UV). The sterilizes insert chip may be reused, for example, by removing the membrane assembly (i.e. membrane+ring and/or reducer) in step 90, and furnishing the insert chip hollow scaffold with a new or refurbished membrane assembly (step 92). Thus, once the Insert-Chip was fabricated it can be easily reused by disassembling the assembled membrane (i.e., membrane with supporting structure).

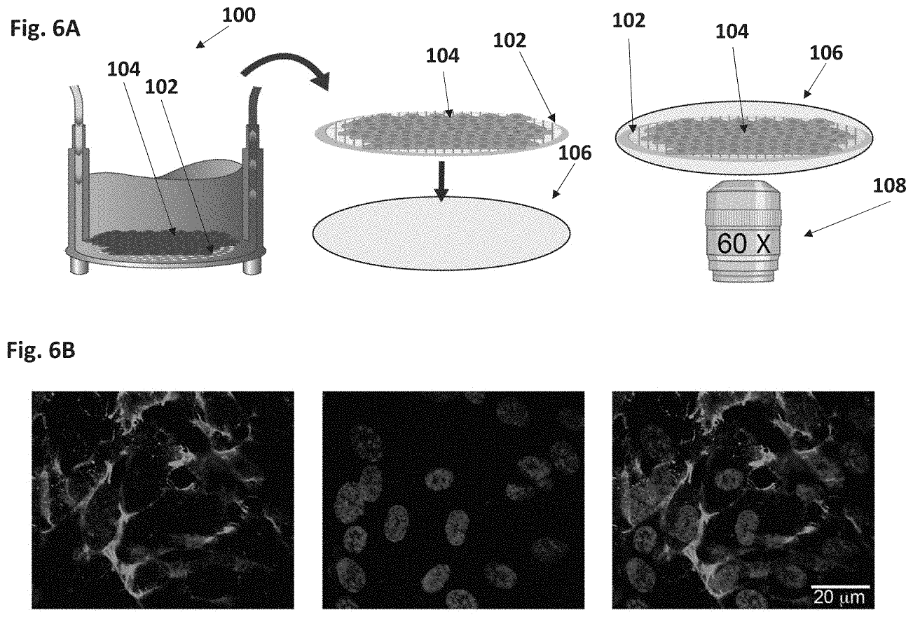

Reference is now made to FIG. 6A, which shows a schematic design (from left to right) of the removal of a porous membrane 102 containing/harboring cultured cells 104 from the insert-chip 100, to be placed on a microscopic cover slip 106, in order to perform high-resolution confocal imaging 108. The results are shown in FIG. 6B, as detailed below herein in the Experimental section.

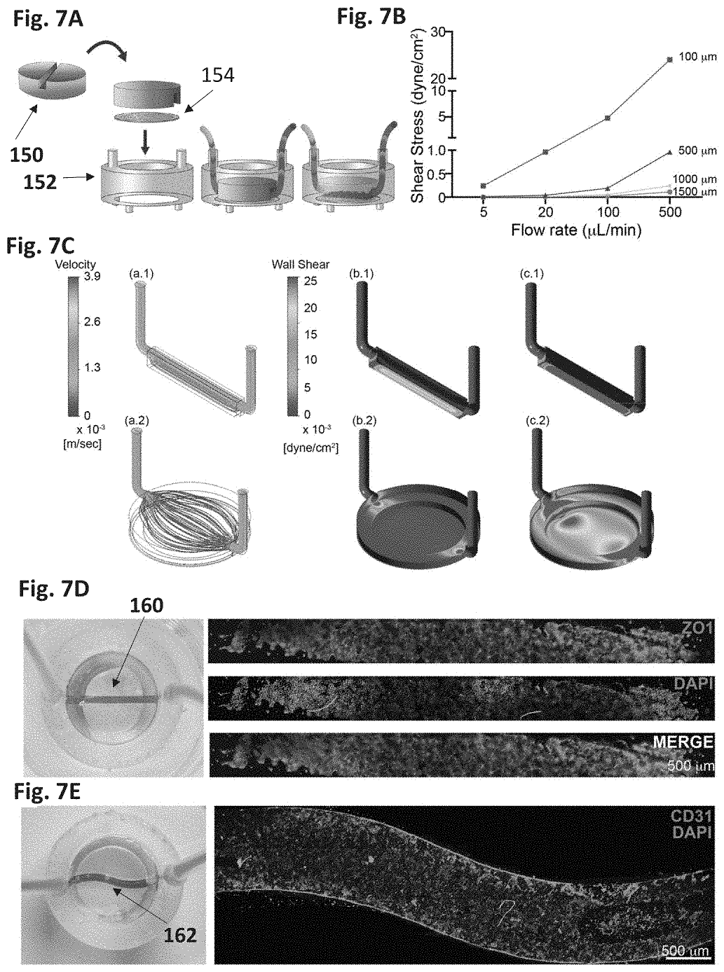

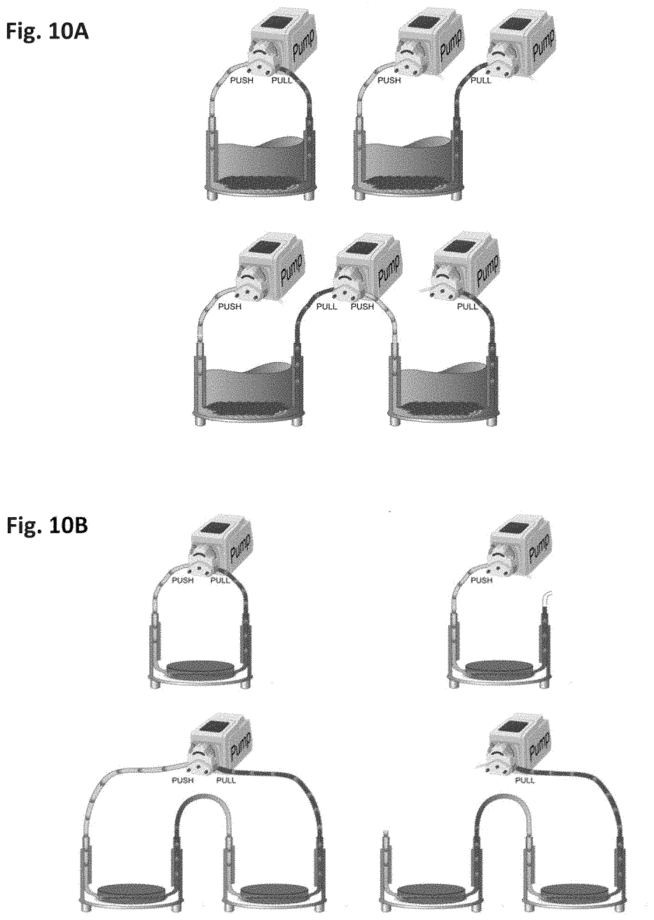

Reference is now made to FIG. 7A which show exemplary insert-reducer, configured to control flow in the insert chip, according to some embodiments. FIG. 7A shows a schematic experimental design of a linear insert-reducer (“reducer”) 150, placed on membrane 154 in chip 152, the reducer enables flow and shear stress to be controlled. Though the basic design of the Insert-Chip allows for the application of flow, and the use of relatively small quantities of cells, to enable the number of cells used to be further reduced, as well as to provide more precise control over the shear forces applied to the cells, an insert-reducer may be used. As detailed above, the “reducer” may be made of PDMS that can easily be placed in the chip, as shown in FIG. 7A. The reducer enables reducing the active surface area, and allowing channels to be created in any desired shape. Moreover, by using the reducers and changing width or other dimensions thereof, it is possible to induce different shear stress, from about 0.001 dyne/cm² to about 30 dyne/cm², depending on the flow rate (as shown in FIG. 7B). Different flow profile can be designed, combining Insert-chip with and without reducer (as illustrated in FIG. 10A and FIG. 10B), with the option to better control the flow and apply the desired shear, even in case in which multiple Insert-Chip are connected. In some embodiments, the flow in the chip is laminar, producing parallel flow lines that wash the entire geometry with no visible flow separation and stagnant regions production a thoroughly perfused system in both the reduced and the non-reduced configurations. In some embodiments, the shear may increase with the flow and can be brought to higher levels in both the reduced and non-reduced configuration. FIG. 7D shows an exemplary linear reducer 160 and FIG. 7E shows an exemplary “s”-shaped reduced 162. In some embodiments, the reducers are PDMS reducers. In some embodiments, the reducers are constructed using a PDMS ring (for example, having 2-25 mm length (for example, 17 mm length), and 0.5-7 mm height (for example, 3 mm height)). In some embodiments, the reducers are constructed with channels in a desired formation integrated into the membrane. In some embodiments, the reducer enables to utilize just 0.5-95% (for example, about 20%) of the whole membrane surface, and thus to suffice with about 5-50% (for example, 15-20%) of the number of cells that would otherwise be needed for a regular well plate or for an insert not including a reducer.

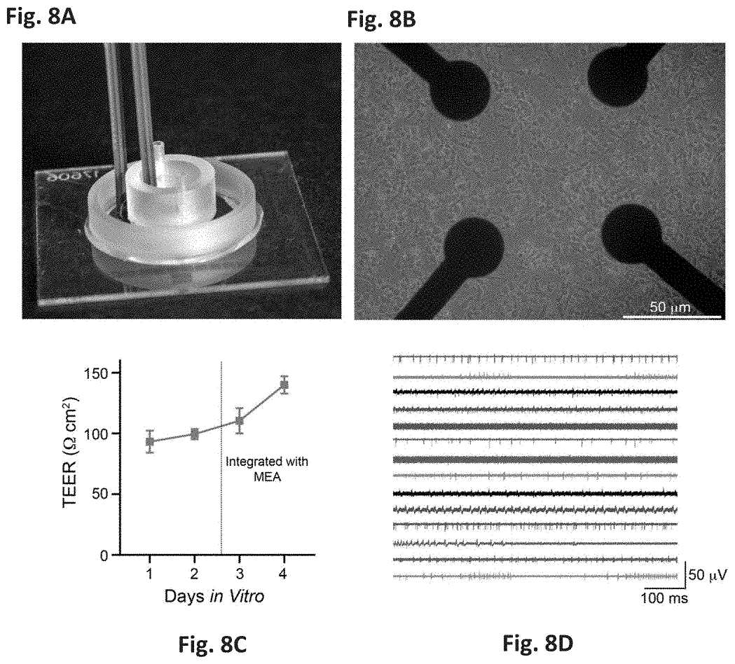

According to some embodiments, as exemplified herein (FIGS. 3A-C, FIGS. 8A-C), the insert chip may be placed/positioned/associate with various cell culture platforms, including, for example, well plates, tissue culture plates, MEA platform, and the like, or any combinations thereof.

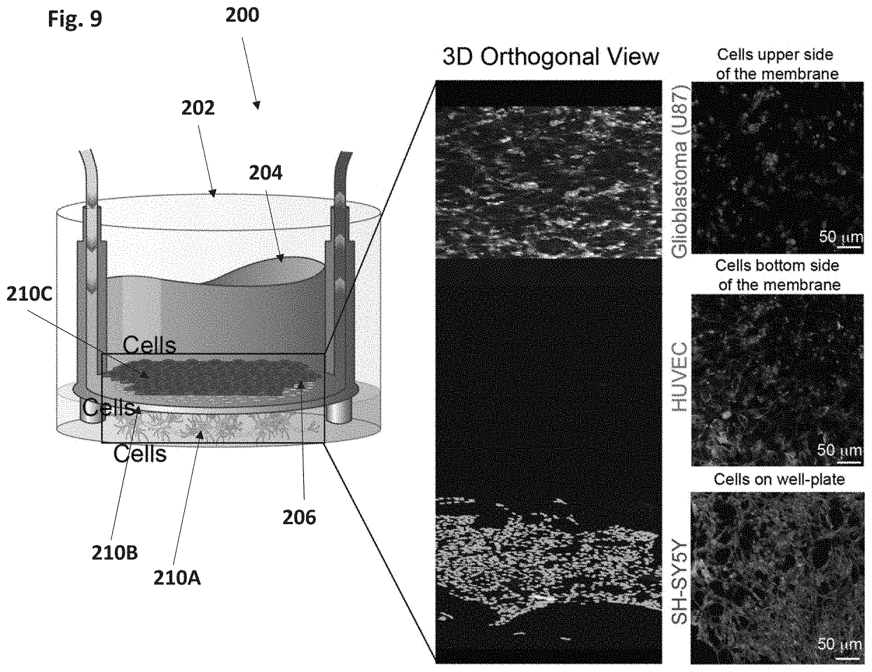

Reference is now made to FIG. 9, which shows an exemplary cell culture system, utilizing an insert chip, according to some embodiments. As shown in FIG. 9, culturing system 200, includes a culture well 202 having placed therein insert chip 204. On the bottom surface of culture well 202, cell population 210A is deployed/grown. On membrane 206 of the insert chip, two cell populations are deployed/growing: cell population 210C on the top surface of membrane 206, and cell population 210B, on the bottom surface of membrane 206 (i.e., the surface that faces the culture well). Thus, the culturing system disclosed herein may be used to simultaneously culture a plurality of different cell populations/cell types and to thereby facilitate studying/testing interaction and cross talk between different cell populations and cell types.

Reference is now made to FIG. 10A which show schematic illustrations of various exemplary flow circulations that can be achieved using the Insert-Chip, depending on the linkage setting of a pump. As shown in FIG. 10A, one or more external pumps (such as, for example, peristaltic pump) may be connected to and between the conduits of the inlets and outlets of the insert chip, thereby facilitating flow direction and or strength between different compartments of a chip and/or between interconnected insert chips. FIG. 10B shows schematic illustrations of various exemplary flow circulations, using a “reduced” Insert-Chip (i.e., utilizing an insert-reducer). Different flow circulations can be achieved using the Insert-Chip with a reducer, depending on the linkage setting of the pump. As shown in FIG. 10B, one or more external pumps (such as, for example, peristaltic pump) may be connected to and between the conduits of the inlets and outlets of the insert chip, thereby facilitating flow direction and or strength between different compartments of a chip and/or between interconnected insert chips.

According to some embodiments, the insert chip is a cylindrical Insert-Chip (as shown, for example, in FIGS. 1A-D and FIG. 2A). According to some embodiments, the insert chip may be a 3D-printed insert chip. According to some embodiments, the insert chip may be formed from a transparent polymer. According to some embodiments, the transparent polymer may be a curable dental resin.

According to some embodiments, each Insert-Chip includes a cell culture chamber (hollow scaffold) having an external diameter customizable to up to 25 mm (for example, in the range of about 2-25 mm). and an inner diameter of 17 mm (for example, in the range of about 1-24 mm), with capacity of up to 2 mL (for example, in the range of about 0.1-4 ml) of fluids (such as, cellular medium or any other suitable buffer). According to some embodiments, the inlet and outlet channels on the upper portion of the chip and/or on lower portion of the chip (if present), enable the chamber to be connected to a controlled flow system (as illustrated, for example, in FIG. 1C and FIG. 1D, and further detailed below). According to some embodiments, the inlet and outlet channels may have any desired length/height, such as, for example, in the range of about 0.5-10 mm. In some embodiments, the inlet and outlet channels length/height is about 5 mm. In some embodiments, with external and internal diameters of 2.5 mm and 1.5 mm, respectively. According to some embodiments, the inlet and outlet openings can be used to connect the chip to a flow system.

According to some embodiments, as discussed herein, the bottom portion of the chip includes one or more (such as, 1-10, for example, 2, 3, 4, 5, 6, 7, 8 or more legs), modular and/or adjustable legs/pillars, which enable the device to be self-standing, while providing visual access to the membrane (e.g., for continuous microscopic visualization of cell growth) and while allowing adjusting the height or distance from the bottom portion of the culture well. According to some embodiments, the insert-chip includes a plurality of pillars/legs/short legs. According to some embodiments, the length of each pillar in the plurality of pillars is within the range of about 0.25 to 10 mm. According to some embodiments, each pillar is about 0.5-4 mm long. According to some embodiments, each pillar is about 0.75-3 mm long. According to some embodiments, each pillar is about 1-2 mm long. According to some embodiments, the plurality of pillars are of the same length. According to some embodiments, the insert chip is configured to stand on the plurality of legs, and thus can stand alone in either a well plate or multi-electrode array (MEA) environment, above a cell culture surface, thereby enabling the cells in that environment to interact with the cells in the chip. According to some embodiments, the height of the legs is adjustable. As used herein, the terms “pillar” and “leg” may interchangeably be used.

According to some embodiments, the membrane is at least partially porous. In some embodiments, the membrane is porous. In some embodiments, the porous membrane is configured to include/harbor/accommodate/hold cells cultured thereon. According to some embodiments, the porous membrane may be positioned near the base of the hollow scaffold, i.e., in close proximity to a lower portion thereof. According to some embodiments, the porous membrane is situated within the hollow scaffold with the support of supporting structure, such as, a PDMS ring. In some embodiments, the membrane can be versatile with respect of composition, size and/or shape. In some embodiments, the membrane may be interfaced to the Insert-Chip with a ring (having, for example an external diameter of about 3-25 mm (for example, about 16 mm) and inner diameter of about 2-24 mm (for example, about 13 mm). To ensure complete adherence between the membrane and the sealing ring, plasma and APTES may be used. According to some embodiments, such process facilitates long-term stability, which is important for reusing the Insert-Chip and for allowing diffusion between the two compartments.

According to some embodiments, the fluid may include any type of suitable fluid, including, for example, but not limited to: buffer, saline, growth medium, tissue culture medium, and the like, or any combination thereof. In some embodiments, different or same fluids may be used/applied for different cells/different cell populations. In some embodiments, specific characteristics of the fluids may be determined according to the performed experiment and/or type of cells. In some embodiments, such specific characteristics of the fluids may include, for example, but not limited to: composition thereof, components thereof, viscosity, ionic strength, inclusion of antibiotics, inclusion of serum, and the like, or any combination thereof.

According to some embodiments, the chip is re-usable, allows for advanced imaging and sensing, and can be used in high-throughput platforms, while providing the enabling to assess organ-organ interactions (see, for example, FIGS. 2A-B).

According to some embodiments, the Insert-Chip has several key design aspects that overcome the current limitations of Organs-on-a-Chip, by leveraging the strengths of “static” TW inserts: The Insert-Chip is a stand-alone platform that can be integrated into almost any standard culturing platform (6, 12 or 24-well plate or MEA substrate) (FIGS. 1A-D and FIGS. 3A-C), and, in doing so, transform it into an Organ-on-a-Chip system. Thus, the insert chip disclosed herein exhibits high compatibility. This feature enables cells to be cultured without undergoing special optimization procedures (in contrast to regular Organs-on-a-Chip). Indeed, cells with different stages of maturation and functionality can be cultured separately on the well plate and on the Insert-Chip. Once the cultures are ready for the experiment, the Insert-Chip can be added. To achieve straightforward integration into standard culture platforms, the Insert-Chip is configured to be self-supported on a plurality of legs (for example, 4 short legs (1-4 mm in length)) with the membrane positioned below the cell culture surface (FIGS. 1A-D) in any suitable orientation design.

According to some embodiments, a key feature of Organs-on-a-Chip is the capacity to accommodate cell-cell interaction and diffusion between compartments. (FIGS. 2A-B, and FIG. 9). To achieve such properties, the Insert-Chip includes a porous membrane that allows the option to create gradient and diffusion between different cell cultures. Furthermore, it allows up to 3 different cell types/populations to be cultured and optionally to interact, within a single experiment (i.e., on top of the membrane, on the bottom of the membrane, and on the bottom of the well, into which the Insert-Chip is inserted (as shown, for example, in FIG. 1C; and FIG. 9)).

According to some embodiments, the insert chip facilitates spatial and/or physical separation of different/distinct/separate population of cells. According to some embodiments, the insert chip facilitates functional association/interaction between different/distinct/separate population of cells. According to some embodiments, the insert chip facilitates spatial and/or physical separation of different/distinct/separate population of cells, while optionally enabling functional association/interaction between the cells.

According to some embodiments, the Insert-Chip can enable different flow configurations (such as shown, for example, in FIG. 10A and FIG. 10B), and/or shear forces (such as shown, for example, in FIG. 2B), to be induced on the cells. It is important to note that, in vivo, epithelial and endothelial cells are constantly subjected to flow, and it is essential for in vitro platforms to recapitulate these conditions.

According to some embodiments, advantageously, the Insert-Chip can be fabricated by a standard 3D printer, using transparent materials such as a PC membrane and clear dental resin, which allow for real-time observations/visualization of cell morphology. Moreover, the advantageously, the membrane can be easily disassembled from the hollow scaffold, enabling cells to be imaged at high-resolution. Notably, this feature also enables the Insert-Chip to be reused (as demonstrated in FIG. 4), making it highly cost-efficient.

According to some embodiments, the insert-chip and systems including the same allows simulating or mimicking various physiological/cellular conditions, which require cell-cell interaction(s), and/or different physiological conditions, such as various follow patterns/conditions, shear forces, and the like. In some embodiments, such physiological/cellular conditions may include, for example, but not limited to: hypoxia (mimicking, for example, various stroke conditions, microbiome conditions, etc.), intra-cellular barriers, cell-cell interactions, tissue-tissue barrier (such as, blood-brain barrier), and the like, or combinations thereof. In some embodiments, as disclosed herein, the use/creation of at least two compartments within the insert chip, allows the study of cellular systems, while not being exposed to external environment (for example, under hypoxia conditions, under different gasses, fluids, and the like).

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. The methods of the disclosure may include a few of the steps described or all of the steps described. No particular step in a disclosed method is to be considered an essential step of that method, unless explicitly specified as such.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. In such embodiments, for example, the statement “the length of the element is equal to about 1 millimeter” is equivalent to the statement “the length of the element is between 0.99 millimeters and 1.01 millimeters”.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

As exemplified herein, a 3D-printed Insert-Chips in different sizes were produced in order to demonstrate their modularity and adaptability to standard cell culture platforms commonly used in a lab. In addition, barrier tissue cells (either endothelial or epithelial cells) were cultured on top of the Insert-Chip membrane and were used to demonstrate the capacity to induce controlled flow in the Insert-Chip and to image cells with high-resolution confocal microscopy. In additional experiments, it has been demonstrated how the chip can be integrated into conventional culturing platforms, while providing the capacity to co-culture cell populations in the presence of flow. To this end, an Insert-Chip cultured with endothelial cells was inserted into an MEA containing parenchymal cells (neurons and astrocytes). Endothelial and neuronal cell functionality was demonstrated via simultaneous barrier and electrophysiological measurements. Finally, experiments with modified versions of the Insert-Chip can further improve the chip's efficiency or suitability for specific types of experiments. The results exhibit the potential and capabilities of the Insert-Chip as a straightforward yet advanced in vitro modeling platform that can benefit both academic and pharmaceutical labs.

### Example 1: Insert-Chip Development, Design and Fabrication

The Insert-Chip was designed using SolidWorks CAD software (SolidWorks Corporation, MA, USA). A schematic representation of the Insert-Chip fabrication is shown in FIG. 4. Prior to printing, model surfaces were checked, and a scaffold was added using PreForm software (PreForm 3.0.1, Formlabs Inc.). Then, the chips were printed in a stereolithography Form2 3D printer (Formlabs, Somerville, Mass.), using a dental long-term (LT) clear resin (Formlabs), with unique mechanical and optical properties. After printing, the chips were washed in isopropyl alcohol (Avantor) in an ultrasound tank, to remove the unreacted resin, and then cured and dried in a UV curing system (Formlabs).

Fabrication and assembly of additional components: SolidWorks CAD software was used to design master-molds for fabrication of the device's additional components: the support structure (PDMS support ring), and two different “reducer” components (insert-reducer) aimed at reducing the active surface area in the chip and controlling the flow. The molds were printed with a commercial polylactic acid filament using a Raise 3D Pro2 Dual Extruder 3D Printer (Raise Technologies Inc., US). Prior to printing, model surfaces were checked, and, if needed, a scaffold was added using Idea Maker software (3.6.1, Raise Technologies Inc, US). Then, the molds were filled with PDMS prepared by mixing Sylgard 184® (Dow Corning, Midland, Mich., USA) with the curing agent at a ratio of 1:10, followed by curing at 60° C. overnight. The resulting PDMS rings and reducers were cleaned in ethanol, dried at room temperature (RT), and then activated in oxygen plasma (Atto-BR-200-PCCE, Diener Electronic, Germany) for 30 s.

Polycarbonate (PC) membranes (0.4 μm pore size, it4ip S.A., Belgium), 25 μm thick, were cut to size with their protective backing on. The protective backings were then removed, and the PC membranes were rinsed with isopropanol, dried under a stream of compressed air, and activated in oxygen plasma for 2 minutes (Diener Electronic, Germany). Then, the membranes were immersed for 30 minutes in 5% aqueous solution of 3-aminopropyltriethoxysilane (APTES, Sigma-Aldrich) in order to introduce amino groups at the surface of the PC membrane. Then they were washed 3 times with water and dried under a stream of compressed air.

PDMS-rings or PDMS-reducers and PC membranes were then aligned and brought into contact, gently pressed together to ensure conformational contact, and baked at 60° C. overnight. The assembled parts were then inserted into the 3D-printed microfluidic Insert-Chip.

The ready-to-use assembled chip was sterilized using 70% ethanol for 30 min, and was then washed with phosphate-buffered saline (PBS, Biological Industries) 3 times and sterilized under a UV lamp for 20 minutes.

Validation of the flow gradient inside the chip: Flow was controlled by an external peristaltic pump (IP-N 8, Ismatec, Cole-Parmer GmbH, Wertheim, Germany), and connections were in elastic tubing (inner diameter 1 mm, outer diameter 3 mm, Ismatec, Germany). The input tube was connected to the inlet of the chip, and the output tube was connected to a reservoir via the peristaltic pump.

### Example 2: Cell Culture

To test the biocompatibility and the versatility of the Insert-Chip, epithelial and endothelial monolayers were cultured separately in Insert-Chips and the cells were monitored under static and flow conditions. Furthermore, in order to demonstrate the significance of the Insert-Chip, cells were also cultured on commercially available Transwells (Corning, USA). Moreover, to demonstrate how the Insert-Chip can be integrated into a more conventional cell culture environment, neuronal cells were cultured in MEAs, in which the Insert-Chip was subsequently placed.

Epithelial culture. For the epithelial model, human epithelial colorectal adenocarcinoma cells (Caco-2 cells, ATCC® HBT-37™, American Type Culture Collection, Rockville, Md., USA) were used. The passages of the Caco-2 cell line ranged from 26^{th} to 40^{th}. After thawing, the Caco-2 cells were cultured routinely in Dulbecco's Modified Eagle Medium (DMEM, Biological Industries), supplemented with 10% heat-inactivated Fetal Bovine Serum (FBS, Biological Industries), 1% Glutamax (Gibco) and 1% Penicillin-Streptomycin-Amphotericin B (PSA, Biological Industries) solution, at 37° C. with 5% CO₂ in a humidifying incubator. Cells were grown to 80-90% confluence before being transferred inside the Insert-Chip. Before seeding, the porous membrane inside the Insert-Chip was treated with Matrigel Basement Membrane Matrix (Corning) used at 1:50 ratio with the culture medium, for 30 min in the incubator. The membrane was then rinsed with culture medium and the Caco-2 cells, harvested with trypsin/EDTA solution (Biological Industries), were seeded at a density of 100.000 cells/cm² and grown for 9-11 days, changing the medium every 4 days of cell culture.

For the flow condition, the tubing was sterilized by perfusing 70% ethanol throughout the entire system at a flow rate of 5 μL/min for 2 hours. Following that, PBS was flushed into the entire system for an additional 2 hours at the same flow rate. Next, the solution containing Matrigel was flowed inside the Insert-Chip to coat the porous membrane, and the device was then incubated for 30 min. After incubation, the device was perfused with cell culture medium, and then the Caco-2 cells were seeded into the Insert-Chip. Next, the entire system was placed in the incubator, and the peristaltic pump was activated to perfuse culture medium at a constant flow rate of 5 μL/min, for 2 days, to ensure the establishment of an intact monolayer of Caco-2 cells.

Endothelial culture. For the endothelial model, Human Umbilical Vein Endothelial cells (HUVEC, PromoCell GmbH, Heidelberg, Germany) were used. After thawing, the HUVEC were expanded in low-serum endothelial cell growth medium (PromoCell), at 37° C. with 5% CO₂ in a humidifying incubator, and used at passage p3-p5. Cells were grown to 80-90% confluence before being transferred inside the device. Before seeding, the PC membrane was treated with Entactin-Collagen IV-Laminin (ECL) Cell Attachment Matrix (Merck) diluted in DMEM (10 μg/cm²), for 1h in the incubator. Then, the HUVEC, harvested using a DetachKit (Promocell), were seeded inside the Insert-Chip at a density of 250.000 cells/cm² and grown for 3-5 days. In the flow condition, the tubing was cleaned and sterilized as described above. Next, the solution containing ECL Matrix was flowed inside the chip and incubated for 1 h, and then cells were seeded. Then, the entire system was placed in the incubator, and the peristaltic pump was activated to perfuse culture medium at a constant flow rate of 5 μL/min, overnight, to ensure the establishment of an intact monolayer of HUVEC.

Cancer cells line. To develop a tri-culture system, cancer cell lines (U87 glioblastoma and SH-SY5Y neuroblastoma cell lines, ATCC®) were used. After thawing, the U87 cells were cultured similarly to the epithelial cells and after reaching 80% confluency, they were seeded on the membrane. The SH-SY5Y cells were cultured in RPMI-F12 Medium (Biological Industries), supplemented with 10% FBS, 7.5% Sodium bicarbonate (Sigma-Aldrich), 1% Glutamax and 1% Gentamycin (Gibco) solution, at 37° C. with 5% CO₂ in a humidifying incubator. Cells were grown to 80-90% confluence before being transferred inside the multi-well plate (Corning, USA), after being harvested with trypsin/EDTA solution (Biological Industries).

Neuronal culture. Primary dissociated cultures were obtained from postnatal rats (p2-p3) as previously described. All experiments were approved by the local authority and performed in accordance with Israeli law. All efforts were made to minimize animal suffering and to reduce the number of animals used. Neuronal hippocampal cells were plated on MEAs (Multi Channel Systems, Reutlingen, Germany) for network investigation. Prior to cell seeding, the MEA substrates were treated with polyethyleneimine (PEI, Sigma-Aldrich) in Borate buffer (Sigma-Aldrich) overnight at 4° C. Then, the substrates were rinsed 4 times with distilled water, sterilized with UV for 1 h and treated with laminin (20 μg/mL, Sigma-Aldrich) diluted in plating medium containing Neurobasal Medium (Gibco), supplemented with FBS (5%, Biological Industries), B27 (2%, Gibco), Glutamax (1%, Gibco) and PSA (1%, Biological Industries), for 4 h, at 37° C.

Neuronal hippocampal cells were then plated on coated MEA substrates in a plating medium and incubated at 37° C. in a humidified atmosphere enriched with 5% CO₂. After 24 h had passed since seeding, the medium was replaced (80%) with serum-free neurobasal medium, supplemented with B27 (2%), Glutamax (1%), PSA (1%) and Gentamycin (1%, Gibco). Culture medium was renewed (50%) every 3 days from seeding. Plating was carried out at a nominal density of 70,000 cells/cm². Cultures were then used for experiments after 9-12 days in vitro (DIV).

### Example 3: Analytical Studies

Computational Fluid Dynamics (CFD) Model. CFD simulations were conducted to characterize the flow in the chip and to determine the influence of the chip LH (leg height) on the diffusion of mass. The fluid volume was derived from the chip geometries corresponding to the reduced and non-reduced configurations for the flow simulations, while a container was added in which the chip is submerged for the diffusion simulations. The geometries were meshed in ANSYS GAMBIT 19 R3 with the final elements number shown in Table 1. All the simulations were conducted in ANSYS fluent 19 R3 using the constant laminar flow assumption for the flow simulations at two flow rates: 5 μL/min and 50 μL/min. The diffusion was modeled through the convection diffusion equation assuming constant diffusivity and mass production rate (see solved equations below). Since there are many configurations possible in the chip, a simple configuration was elected where the cells are located at the bottom of the reduced container producing CO₂ at an arbitrary constant rate (0.0054 mmol/m²/sec) while there are no cells anywhere else and there is no membrane. The CO₂ diffusivity was taken to be 2.3e⁻⁹ m²/sec and only one flow rate of 5 μL/min was used. Finally, both steady state simulations to derive the final concentration gradients in the chips as well as transient simulations for 360-time steps of 1 second (6 minutes total) were performed to estimate the time scales involved and to produce movies of the diffusion process.

TABLE 1
Mesh types and number of elements

| Geometry | Element Type | Number of Elements |
|---|---|---|
| Non-Reduced | Tetrahedral, Prism | 1M |
| Reduced | Tetrahedral, Prism | 300K |
| 1 mm legs | Tetrahedral | 400K |
| 4 mm Legs | Tetrahedral | 400K |

Solved Equations:

Momentum

∂/∂t(ρv\vec{ })+∇(ρv\vec{ }v\vec{ })=−∇p+∇·(t⁼)+ρg\vec{ }+F\vec{ } (eq. 1)
p—static pressure
ρg\vec{ } and F\vec{ }—gravitational body force and external body forces, respectively.

τ⁼=μ[(∇v\vec{ }+v\vec{ }\hat{ }T)−⅔∇·v\vec{ }I] (eq. 2)
μ—molecular viscosity
I—unit tensor,
⅔ ∇·v\vec{ }I—volume dilation.

### Continuity:

dρ/dt+∇·(ρv\vec{ })=0 (eq. 3)

### Wall Shear Stress:

τ_w=μ∂u/∂n (eq. 4)
u—near wall velocity vector field.
n—wall normal vector.

### Convection Diffusion

∂c/∂t=∇·(D∇c)−∇·(uc)+R (eq. 5)
Where c is concertation, D is the diffusivity coefficient in water, u is the velocity field obtained from equation 1 and R describes sources or sinks.

Fixation, immunocytochemistry, and confocal imaging—HUVEC, Caco-2 and the cancer cells lines were rinsed in PBS and fixed in 4% paraformaldehyde (PFA, Sigma-Aldrich) for 20 minutes at RT. Immunocytochemistry was carried out after permeabilization with 0.1% Triton X-100 (Sigma-Aldrich) in PBS for 10 min at RT and blocking for 30 min in FBS (5%) in PBS. Primary antibodies were applied overnight in PBS at 4° C. The following primary antibodies were used for immunocytochemistry experiments: rabbit anti-ZO-1 (Abcam) and rabbit anti-CD-31 (Abcam), to stain the zona occludens-1 (a key component of tight junctions) in Caco-2 cells and the endothelial cell adhesion molecule 1 in HUVEC, respectively; mouse anti-GFAP (Abcam), to stain the Glial Fibrillary Protein in U87 cells; Phalloidin-iFluor 488 (Abcam), to stain actin in SY-SY5Y cells. Cells were then washed three times in PBS and stained with the secondary antibody for 1 h at RT. The secondary antibodies were anti-rabbit Alexa Fluor-488 (Invitrogen) and anti-mouse Alexa Fluor-594 (Invitrogen). After being washed four times with PBS, cells were mounted on a 0.17-mm-thick glass coverslip using DAPI-Fluoromount-G® (SouthernBiotech), to stain the nuclei. Imaging was carried out using an inverted confocal microscope (Olympus FV3000-IX83), with appropriate filter cubes and equipped with 2×/0.08 NA, 10×/0.3 NA, 20×/0.8 and 60×/1.42 NA objectives. For imaging the entire channel within the PDMS-reducer, images were acquired by sequential tile scanning. Image reconstruction and processing were done using open-source ImageJ software.

Trans-epithelial endothelial electrical resistance (TEER). The barrier properties of the epithelial/endothelial monolayer were evaluated with TEER measurements along the cellular growth period. TEER was measured with the Millicell ERS-2 Voltohmmeter (Merck Millipore). TEER values (MΩ cm²) were calculated and compared to those obtained in an Insert-Chip not containing cells, considered as blank, and were obtained from 4 different individual experiments, with 2 Insert-Chips used in each experiment.

Permeability Assay. HUVECs and Caco-2 were cultured on the Insert-Chip in static and under-flow condition. Permeability of the monolayer was assessed by measuring leakage of Fluorescein isothiocyanate (FITC)-dextran (Sigma-Aldrich) administered to the upper compartment of the Insert-Chip at different time points. One hour after adding dextran, the fluorescence intensity of the medium in the lower compartment was measured by a fluorescent plate reader (Multiskan Go, Thermo Scientific), at an excitation of 492 nm and emission of 518 nm (2 Insert-Chip for each condition)

MEA recording. Neuronal network extracellular recordings were carried out using the MEA60 system (Multi Channel Systems). Primary hippocampal cultures were plated on Titanium Nitride (TiN) MEAs with 60 electrodes (30 μm dimeter, 200 μm inter-electrode spacing). Raw data were monitored and recorded by using the commercial software MCRack (Multi Channel Systems), at 37° C., in the presence of cell culture medium. The recorded events were analyzed offline with NeuroExplorer 5.127 software (Nex Technologies, Colorado, USA).

Statistical analysis. The results are presented as the mean±SD. Statistically significant differences among multiple groups were evaluated by two-way ANOVA, followed by the Holm-Sidak test for multiple comparison (GraphPad Prism 8.4.3). A statistically significant difference between two data sets was assessed and P<0.05 was considered statistically significant.

### Example 4: Insert-Chip Fabrication

Most Organs-on-a-Chip or microfluidic devices are fabricated from PDMS, which is biocompatible, transparent, and has good gas permeability. However, a major limitation of PDMS is its hydrophobicity, which causes substantial absorption of hydrophilic materials. Moreover, in some cases, chip fabrication requires specific knowhow and facilities. To overcome these challenges, stereolithograpy 3D printing was used for fabricating the Insert-Chip. The use of 3D-printing enables the design of the desired platform to be quickly modified, and it reduces the need for multi-step fabrication needed in “standard” Organs-on-a-Chip. Furthermore, the use of 3D-printing reduces the fabrication time of the Organ-on-a-Chip from several days to a few hours, as well as the possibility to use not-absorbing materials. The Insert-Chip is made only from 3 parts (FIG. 1A): Base (hollow scaffold), membrane and sealing/supporting element (ring). The base is fully made with a 3D printer (as detailed above). The membrane can be versatile, i.e., there are no restrictions on what material can be used. In this example, porous PC was used for the membrane (0.4 μm pore size) (FIG. 1C). The membrane is interfaced to the Insert-Chip with a ring (16 mm external and 13 mm inner diameter) made of PDMS, previously fabricated in a specific 3D-printed mold (see Example 1, for details). To ensure complete adherence between the membrane and the sealing ring, plasma and APTES were used. This process ensures long-term stability, which is crucial for reusing the Insert-Chip and for allowing diffusion between the two compartments, as demonstrated in FIG. 1D, using different color solutions.

### Example 5: Insert-Chip Modularity and Compatibility with Standard In Vitro Platforms

An important feature of the Insert-Chip is the fact that “one-design fits all”, i.e., the chip is modular and can be integrated with existing platforms. One of the strengths of the “standard” dual-channel Organ-on-a-Chip platform is that it provides the capacity to observe cell-cell interactions. With the Insert-Chip, cell-cell interactions can take place between the cells plated on the insert chip membrane and the cells cultured in the well into which the device is inserted. The characteristics of these interactions are mainly determined by the flow rate, pore size of the membrane, and the distance between the two cell populations (the distance between the membrane and the bottom of the plate). As the Insert-Chip is fabricated via 3D printing, all these parameters can be adjusted in accordance with experimental requirements. For example, FIG. 2A shows an example in which the length of the Insert-Chip's legs is adjusted to change the distance (height) between the membrane and the bottom plate. This versatility is especially important for controlling the diffusion, material gradient, and shear forces between the upper and lower compartments. For proof of principle, Insert-Chips were fabricated with 3 different heights (FIG. 2A), 1 mm, 2 mm, and 4 mm and the diffusion of CO₂ was simulated in the Insert-Chip with 1 mm and 4 mm legs-height (LH) (FIG. 2B). The diffusion simulations show that the general influence of the chip LH is to control the relative influence of convective vs. the purely diffusive mass transport with increased LH. This trend is demonstrated by the stable diffusive front in the 4 mm LH configuration where the reducer flow chamber is relatively far from the CO₂ producing cells at the bottom and does not induce a significant convective transport in the container. Thus, even at infinite time, there is no CO₂ in the reducer, and the concentration at the bottom will continue to rise unhindered until saturation (FIG. 2B). On the other hand, when the reducer is closer to the source of the CO₂ the entire distribution map is skewed toward the outlet resulting eventually in removal of mass through the outlet when the system reaches a steady-state (FIG. 2B). This trend will vary in intensity in different system configurations but will always be present due to the low pressure created by the flow, even when the membrane will be in place. The capacity to insert the chip into almost any standard in vitro platform (e.g. 6, 12 and 24 well plates, as shown in FIG. 3A) is a key benefit for biomedical experiments, as this feature contributes towards cost-efficiency, reduces the need for customized equipment, and enables high-throughput systems (e.g., 24-well plates) to be used as “dual-compartment Organs-on-a-Chip”.

Moreover, when multiple Insert-Chips are placed next to each other (FIG. 3A), it is possible to link/connect them together (FIG. 3B and FIG. 3C), thus creating multi-organ-chip systems. Furthermore, two different cell types can be cultured on each side of the membrane and placed in contact with cells grown in another support, such as a well-plate, which creates a tri-culture system as shown in FIG. 9.

This feature can contribute substantially to the study of human physiology and pharmacokinetics and pharmacodynamics, for which organ-organ interactions are crucial, yet highly challenging to mimic in vitro.

### Example 6: Endothelial and Epithelial Barriers

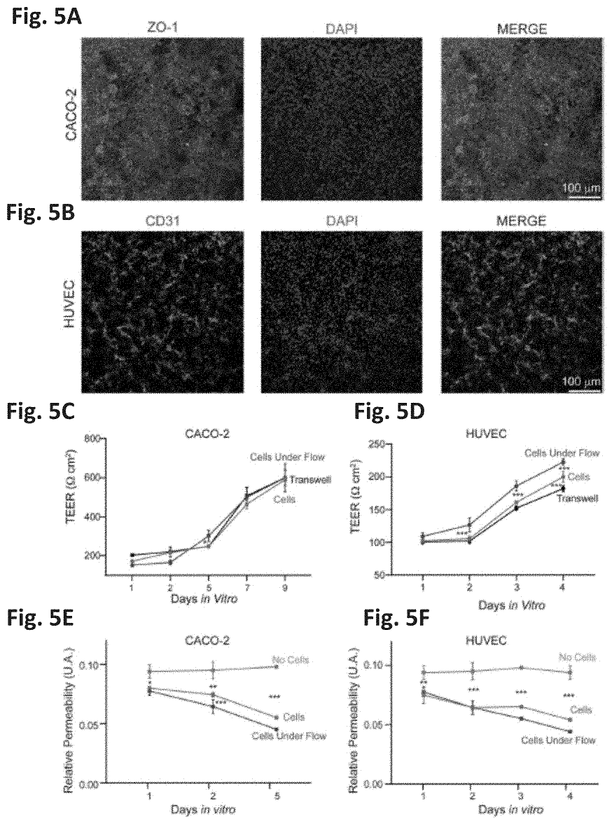

The aim of the study was to demonstrate the use of the Insert-Chip as a modular “Epithelium-on-a-Chip” (Caco-2 cells) or “Endothelium-on-a-Chip” (HUVEC) (FIG. 5A and FIG. 5B). These cell types were selected because all parenchymal tissues interact with barrier tissues, and it is known that these tissues show better properties under flow, and the capacity to induce controlled flow is one of the strengths of the system.

Cell growth and barrier development was monitored over 4 and 9 days (from 1 to 4 or from 1 to 9 DIV), until the Caco-2 cells and HUVEC formed complete confluent monolayers (FIG. 5C 5D, respectively). Once the cells showed confluent monolayers, barrier function was further tested via immunocytochemistry (FIGS. 5A and 5B), demonstrating a continuous distribution of tight junctions in both cellular types. In addition, both TEER and permeability measurements were used to assess barrier function over the course of the observation period (FIGS. 5C to 5F).

Both methods provide complementary information on the barrier properties, as TEER provides a quick, non-invasive and real-time indication of barrier properties; while fluorescent assays can provide information on how the permeability changes with the molecular weight; it is important to note that the design of the Insert-Chip allows for the use of commercial TEER systems. TEER measurements were used to compare our Insert-Chip system to the ones measured on commercially available Transwells.

No significant differences were found in Caco-2 cells cultured under flow (from 151.2±6.2 Ωcm² to 600.0±70.7 Ωcm²) compared to the ones grown without flow (from 171.2±6.2 Ωcm² to 590.0±11.5 Ωcm²) or on Transwells (from 202.5±5.01 cm² to 600.5±40.0 Ωcm², FIG. 5C). Conversely, when comparing HUVEC cells, significant differences were found between cells grown in the Insert-Chip under flow (from 109.2±5.6 Ωcm² to 222.5±5.0 Ωcm²) to the ones without flow (from 102.5±2.8 Ωcm² to 200.0±8.1 Ωcm²) or on Transwells (from 100.7±2.9 Ωcm² to 182.2±4.5 Ωcm²; FIG. 5D).

To validate the culturing system/platform, permeability measurements were done without cells and cells that were cultured with and without flow. This was performed by quantifying the rate at which water soluble fluorescein isothiocyanate (FITC)-dextran was transported across the endothelium and epithelium to the bottom compartment of the Insert-Chip upon addition at the upper one (FIGS. 5E and 5F). Significant decrease in terms of absorption measurement after 1, 2 and 5 days for the Caco-2 cells (FIG. 5E) and after 1, 2, 3 and 4 days for the HUVEC cells (FIG. 5F) (in static and under flow condition) confirmed the establishment of cellular barriers, compared to Insert-Chip without cells. In other words, it can be seen that cells created a barrier layer, and that flow induction enhanced the barrier properties.

### Example 7: High-Resolution Imaging Capabilities

High-resolution imaging is an indispensable tool for studying the structure and the dynamics of cells. Unfortunately, it is highly challenging to do high-magnification imaging with “standard” dual-channel Organs-on-a-Chip, as the typical working distance of 40×, 60× objectives is 170-200 μm, and the distance of the membrane where cells are cultured from the bottom of the Chip is usually above 300 μm. To overcome this challenge, the Insert-Chip was designed such that the membrane can be easily removed from the chip (FIG. 6A) after the culture period, due to the presence of the PDMS ring, by, for example, using a tweezer. Once the membrane is removed, it can be placed on a glass coverslip and standard immunocytochemistry can be performed on the membrane, which can be mounted onto a glass slide for high-magnification imaging (FIG. 6A). As shown in FIG. 6B, high magnification (60× oil objective) of HUVEC, stained for CD-31 protein (in green) and DAPI (blue) for the nuclei, enables cell junctions to be better identified and investigated.

### Example 8: Chip Reducer and Shear Force Application

Though the basic design of the Insert-Chip allows for the application of flow, and the use of relatively small quantities of cells, it was sought to enable the number of cells used to be further reduced, as well as to provide more precise control over the shear forces applied to the cells. To do so, a “reducer” (insert-reducer) made of PDMS that easily can be placed in the chip (FIG. 7A), reducing the active surface area, and allowing channels to be created in any desired shape (FIGS. 7A-E). Moreover, by using the reducers and changing their width, it is possible to induce different shear stress, from 0.001 dyne/cm² to almost 30 dyne/cm², depending on the flow rate (see plot in FIG. 7B). Different flow profile can be designed, combining Insert-chip with and without reducer (FIG. 10A and FIG. 10B), with the possibility to better control the flow and applied the desidered shear, even in case in which multiple Insert-Chip are connected. To better characterize the flow profile of the Insert-Chip, computational simulation were performed. The flow in the chip is laminar, producing parallel flow lines that wash the entire geometry with no visible flow separation and stagnant regions production a thoroughly perfused system in both the reduced (FIG. 7C(a.1)) and the non-reduced (FIG. 7C(a.2)) configurations. Although the system does develop a non-negligible Wall Shear Stress (WSS) at the 5 μL/min it is significantly below any physiological shear stress that endothelial cells in the human body are normally exposed to.

Nevertheless, the shear increases with the flow and can be brought to higher levels in both the reduced (FIG. 7c(c.1)) and non-reduced configuration (FIG. 7c(c.2)). It can also be seen that in the reduced (FIGS. 7c(c.1) and 7c(b.1)) configuration, the WSS is more uniform than in the non-reduced system (FIGS. 7c(c.2) and 7c(b.2)), requiring the use of a reducer to produce uniform conditions.

In these experiments, two reducers were used, one with a linear shape (FIG. 7D) and one with an “S” shape (FIG. 7E). Both reducers are constructed using a PDMS ring (17 mm length, 3 mm high) with channels in the desired formation integrated into the membrane. The reducer enables a user to utilize just 20% of the whole membrane, and thus to suffice with 15-20% of the number of cells that would be needed for the basic version of the Insert-Chip, or for a regular well plate.

To demonstrate the use of the reducer, Caco-2 cells were cultured in the Insert-Chip with the linear shape reducer (FIG. 7D), and HUVEC were cultured in the “S” shape reducer (FIG. 7E) In order to achieve confluency, the cells were under a constant flow rate of 5 μL/min, for 2 days. It can be seen that the Caco-2 (FIG. 7D) and the HUVEC (FIG. 7E) cells established adherens junctions in the epithelial and endothelial monolayer, indicating successful establishment of an intact barrier.

### Example 9: Integrated TEER and MEA Measurements

As most of the parenchyma is surrounded by barrier layer, there is a need for creating such co-culture systems which allow to culture barrier layer and parenchymal, while assessing their functionality. an Organ-on-a-Chip with multiple sensors, in which it is possible to simultaneously measure both barrier function via TEER and the electrical activity of excitable cells, using MEAs was demonstrated by Maoz et al., Lab Chip, 17: 2944, 2017. However, this platform requires custom fabrication and is therefore less accessible than commercial platforms. The Insert-Chip was designed to overcome this challenge; that is, it can be integrated into a commercial MEA platform (FIGS. 8A-C), such that permeability of the barrier tissue can be measured using a commercial TEER system, while the electrical activity of excitable cells is measured using the commercial MEA platform (FIG. 8A). To demonstrate this capability, the neurovascular system was used as an example for such use. The blood brain barrier is a protective layer to the neurons which is the brain parenchymal. HUVEC were cultured on the Insert-Chip; when the cells created a confluent monolayer, the chip was placed on top of a commercial MEA plate cultured with hippocampal neurons (FIG. 8B). Barrier permeability was monitored with TEER (FIG. 8C), together with neuronal electrical activity (FIG. 8D), which remained robust over 10-12 DIV, giving the possibility to simultaneously monitor both cellular functionalities even if characterized by different maturation times.

It is to note that such experiments are challenging to carry out with “standard” Organs-on-a-Chip, not only because of the technological aspect but also because of the biological aspect, which requires that both cell populations be at the same stages of maturation and functionality, which might be hard to coordinate. For example, it takes 1-3 days for the HUVEC to create a fully functional barrier; however, it takes at least 10 days to achieve robust neuronal activity. Use of the Insert-Chip enables the experimenter to culture each of the cell populations separately, and to combine them-by inserting the Insert-Chip into the MEA plate-only when both populations are mature.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An insert chip for cell culture, the insert chip comprises:
a. a hollow scaffold adapted to enclose therewithin a porous membrane, wherein the hollow scaffold comprises at least one inlet configured to deliver a fluid thereinto and at least one outlet configured to withdraw fluids therefrom and wherein each of said at least one inlet and at least one outlet is configured to fluidly associate with a corresponding fluid receptacle, through corresponding conduits;
b. the porous membrane having an upper side and a lower side, and is configured to accommodate cell culture on each side thereof, wherein the porous membrane is configured to be positioned at a lower portion of the hollow scaffold; and
c. one or more legs extending from a bottom part of the hollow scaffold and configured to position the hollow scaffold on a surface such that the distance between the lower side of the porous membrane and the flat surface is determined by the length of the plurality of legs.

2. The insert chip according to claim 1, further comprising a support structure, configured to support the porous membrane within the hollow scaffold.

3. The insert chip according to claim 1, wherein the length of the one or more legs is adjustable.

4. The insert chip according to claim 1, wherein said inlet and said outlet are positioned in the wall of the hollow scaffold.

5. The insert chip according to claim 1, wherein the membrane is associated with an inner surface of the hollow scaffold.

6. The insert chip according to claim 1, wherein the membrane is at least partially held between inner walls of the hollow scaffold.

7. The insert chip according to claim 1, wherein the membrane is removable.

8. The insert chip according to claim 1, further comprising an insert-reducer configured to reduce a surface area of the membrane on which the insert-reducer is positioned.

9. The insert chip according to claim 1, comprising two inlets and two outlets.

10. The insert chip according to claim 9, wherein each set of inlet and outlet is configured to allow passage of a separate fluid.

11. The insert chip according to claim 9, wherein each set of inlet and outlet is configured to provide fluid flow to a distinct compartment of the insert chip.

12. The insert chip according to claim 1, configured to allow controlling or determining flow pattern, flow strength and/or shear forces applied on cells associated therewith.

13. The insert chip according to claim 1, wherein the chip is configured to be placed or located in a culture well plate and/or multi-electrode array (MEA).

14. The insert chip according to claim 2, wherein the support structure is placed on at least a portion of the top surface of the membrane.

15. A cell culture system comprising
at least one cell culture container comprising an insert chip according to claim 1, wherein the porous membrane is positioned within the insert chip such that the lower side thereof is facing the bottom surface of the at least one cell culture container, and is detachable therefrom;
at least one first fluid receptacle fluidly associated with the at least one inlet; and
at least one withdrawn fluid receptacle fluidly attached to the at least one outlet.

16. The cell culture system of claim 15, further comprising at least one inlet conduit fluidly connecting the at least one inlet to the at least one fluid receptacle; and at least one outlet conduit fluidly connecting the at least one outlet to the at least one withdrawn fluid receptacle.

17. The cell culture system of claim 15, wherein said at least one cell culture container is configured to contain fluids at a fluid level such that the fluids contact the porous membrane, wherein the fluids include a fresh first fluid flowing therein through the at least one inlet and withdrawn therefrom through the at least one outlet at a flow rate adjusted to maintain the fluids level within the cell culture container.

18. The cell culture system according to claim 15, configured to allow essentially simultaneous culturing of at least two distinct cell populations.

19. The cell culture system according to claim 18, wherein the distinct cell populations are physically and/or spatially separated.

20. The cell culture system of claim 15, wherein said insert chip comprises two inlets and two outlets, wherein each set of inlet and outlet is configured to provide fluid flow to a distinct compartment of the insert chip.
